(19) United States
(12) Patent Application Publication
(10) Pub. No.: US 2009/0275871 A1
(43) Pub. Date: Nov. 5, 2009

(54) BACK BRACE FOR SPINAL CORRECTION AND ITS MANUFACTURING METHOD

(76) Inventor: Gerald Liu, Tainan City (TW)

Correspondence Address: Gerald LIU, P. O. BOX 90, Tainan City, 70499, TW

(21) Appl. No.: 12/113,951

(22) Filed: May 2, 2008

(51) Int. Cl. A61F 5/00

(52) U.S. Cl. 602/19; 602/20; 602/36

(57) ABSTRACT

The invention relates to a back brace for spinal correction and its manufacturing method. A correction brace is made according to a patient's shape, having its inside installed with plural air bags corresponding to a deformed portion and locations of reverse spinal rotation. Each air bag has an inflation valve fitted in a small through hole bored in the correction brace for a professional to inflate or deflate it to adjust pressure, laid with a soft protection cushion. An effective correction of scoliosis and reverse rotation of the vertebra can be achieved by the air bags correspondingly installed inside the correction brace to force a spinal deformed portion in three dimensions.

### BACKGROUND OF THE INVENTION

1. Field of the Invention

This invention relates to a back brace for spinal correction and its manufacturing method, particularly to one provided with air bags fixed inside a correction brace, able to correct serious scoliosis and rotation of a vertebra of a spine by coupling with other medical treatments.

2. Description of the Prior Art

Usually, if a human being has scoliosis or other spinal deformities, he/she should be diagnosed and treated by experts, coupled with rehabilitation treatment to keep his/her spine normally re-positioned. According to Cobb angle, a scoliosis curve less than 20 degrees usually means that nothing needs to be done except for regular checkups and rehabilitation; if the curve is 20 to 40 degrees, except rehabilitation, to wear a back brace is generally suggested; as for a curve larger than 40 degrees, surgery is necessary because rehabilitation and wearing a back brace does not work effectively. However, as surgery is supposed to be risky and involve sequelae, it is always the last choice for a spinal correction. Therefore, non-surgical rehabilitation and wearing a back brace are usually a priority of spinal correction.

As shown in FIG. 1, a conventional back brace 1 is provided with a main body 10 made of plastic in shape of a user, an opening 11 formed while pulling open outwards the main body 10 for a user to wear it on, a plurality of fastening belts 12 positioned on two corresponding sides of the opening 11 for securing it on a user's body, a vertical supporter 13 located at one side of the main body 10 for fixing a curved body of a patient, and an elastic belt 14 employed to keep the main body 10 fixed on a patient, worn on a patient to keep spine positioned to prevent the spine from re-deformed. But, the conventional back brace 1 has disadvantages mentioned below.

1. The conventional back brace 1 can only function to fix and support the spine so as to prevent the spine from continuing to further curve; once it is taken off a patient, curvature of the spine may again get worse, unable to really achieve a correction.

2. As a standard wearing time of the back brace 1 has to last 23 hours each day, it is prone to make a wearer feel uncomfortable.

3. To wear the back brace 1 for such a long period of time is to gradually pose hardening or atrophy of the back muscles.

4. The back brace 1 is only suitable for correcting a deformed angle less than 40 degrees, with no capability of correcting spinal rotation.

5. As the back brace 1 has to be worn on clothes, it makes a wearer look unaesthetic, possible to lower patients' willingness to wear it.

### SUMMARY OF THE INVENTION

The object of this invention is to offer a back brace for spinal correction and its manufacturing method. The back brace can be worn quickly, and will not make a wearer look clumsy and cause atrophy of the back muscles. Especially, a patient who has a curved angle more than 40 degrees, conventionally needing a surgery to correct it, can wear the back brace to positively achieve an effective correction for scoliosis and reverse rotation of the spine.

The main characteristics of the invention are a correction brace, plural air bags and protection cushions. A patient's spine is previously photoed by X-ray to identify its deformity. An original gypseous model is made according to a patient's body shape with a curvature. The original gypseous model is next amended to become a revised model in a normal shape by filling a recessed portion with gypsum and cutting off a humped portion. The revised model is successively wrapped with a heated thermoplastic board that becomes the correction brace after cooled down. The correction brace is provided with a vertical opening cut in its rear side for being pulled wide open to enable a patient to wear it on, and plural locking members correspondingly positioned at two sides of the vertical opening for adjusting the width of the vertical opening and the tightness of the correction brace. The air bags have different sizes, fixed on an inner wall of the correction brace to correspond to a humped portion of a scoliosis, an upper point and a lower point of the humped portion, and a location of a rotated vertebra. Each the air bag is provided with an inflation valve fitted in a small through hole bored in the correction brace for a professional to inflate or deflate it without necessity of undressing the back brace, and laid with the soft protection cushion. So, an effective correction of scoliosis and reverse rotation of the vertebra can be achieved by the air bags correspondingly installed inside the correction brace to force a spinal deformed portion in three dimensions.

### BRIEF DESCRIPTION OF DRAWINGS

This invention is better understood by referring to the accompanying drawings, wherein:

FIG. 1 is a perspective view of a conventional back brace;

FIG. 2A is a simple illustrative view of an original gypseous model made according to a patient's shape in a preferred embodiment of a back brace for spinal correction and its manufacturing method in the present invention;

FIG. 2B is a simple illustrative view of a revised model made by amending the original gypseous model in the preferred embodiment of a back brace for spinal correction and its manufacturing method in the present invention;

FIG. 2C is a simple illustrative view of a revised model wrapped with a heated thermoplastic board so as to make a correction brace in the preferred embodiment of a back brace for spinal correction and its manufacturing method in the present invention;

FIG. 3 is an exploded perspective view of the preferred embodiment of a back brace for spinal correction and its manufacturing method in the present invention;

FIG. 4 is a perspective view of the preferred embodiment of a back brace for spinal correction and its manufacturing method in the present invention, showing air bags installed inside the correction brace;

FIG. 5 is a rear perspective view of the preferred embodiment of a back brace for spinal correction and its manufacturing method in the present invention;

FIG. 6 is a front perspective view of the preferred embodiment of a back brace for spinal correction and its manufacturing method in the present invention;

FIG. 7 is a simple illustrative view of the preferred embodiment of a back brace for spinal correction and its manufacturing method in the present invention, showing it being worn on a patient with the air bags not inflated;

FIG. 8 is a simple illustrative view of the preferred embodiment of a back brace for spinal correction and its manufacturing method in the present invention, showing it being worn on a patient with the air bags inflated;

FIG. 9 is a simple illustrative view of the preferred embodiment of a back brace for spinal correction and its manufacturing method in the present invention, showing it being worn on a patient having a curved spine located around the chest;

FIG. 10 is a simple illustrative view of the preferred embodiment of a back brace for spinal correction and its manufacturing method in the present invention, showing it being worn on a patient having a curved spine located around the waist;

FIG. 11 is a simple illustrative view of the preferred embodiment of a back brace for spinal correction and its manufacturing method in the present invention, showing it being worn on a patient having an S-shaped scoliosis;

FIG. 12 is a simple illustrative view of the preferred embodiment of a back brace for spinal correction and its manufacturing method in the present invention, showing it being worn on a patient having a scoliosis coupled with spinal rotation;

FIG. 13 is a top cross-sectional view of the preferred embodiment of a back brace for spinal correction and its manufacturing method in the present invention, showing it being worn on a patient having a scoliosis coupled with a spinal rotation around the chest; and

FIG. 14 is a top cross-sectional view of the preferred embodiment of a back brace for spinal correction and its manufacturing method in the present invention, showing it being worn on a patient having a scoliosis coupled with a spinal rotation around the waist.

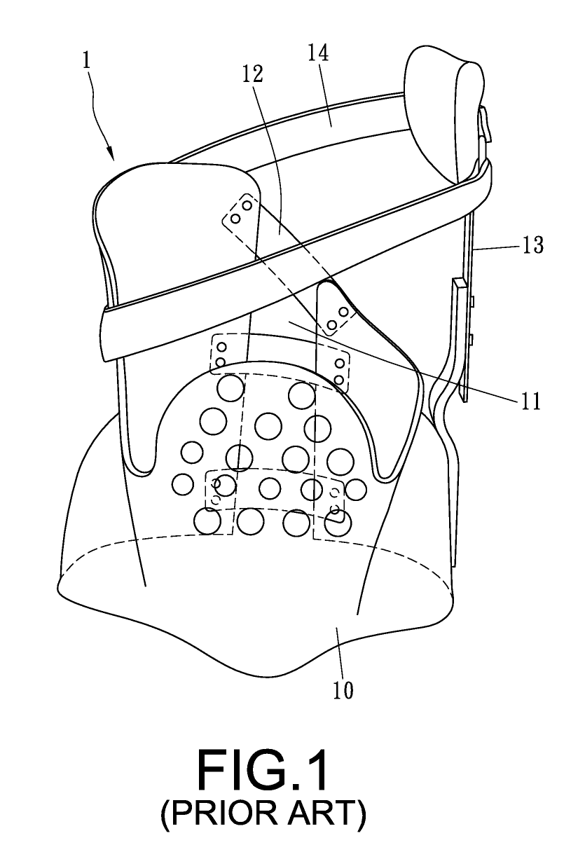

### DETAILED DESCRIPTION OF THE PREFERRED EMBODIMENT

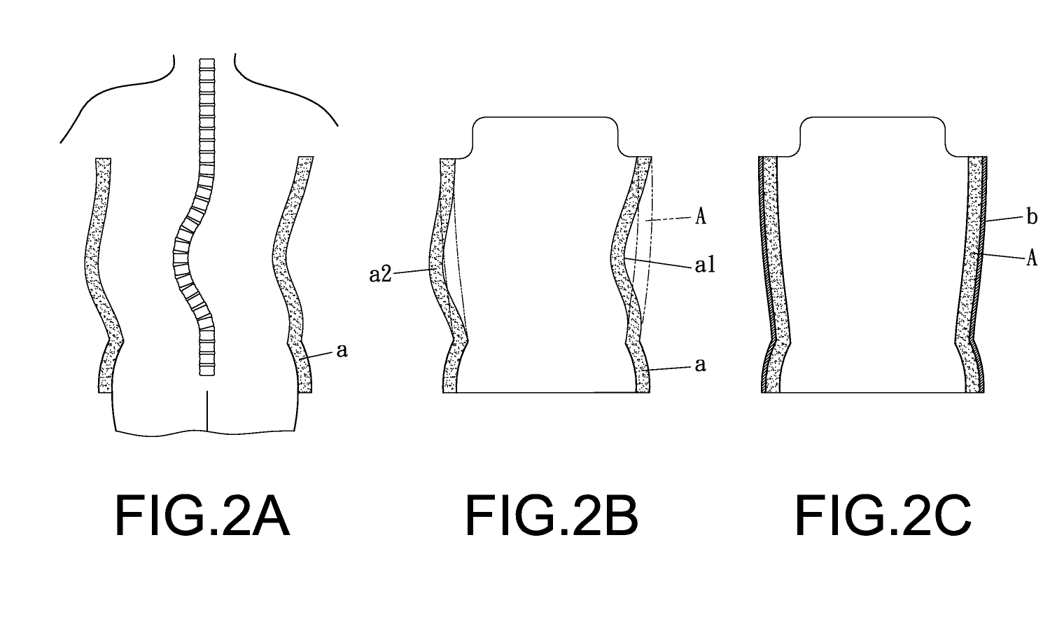

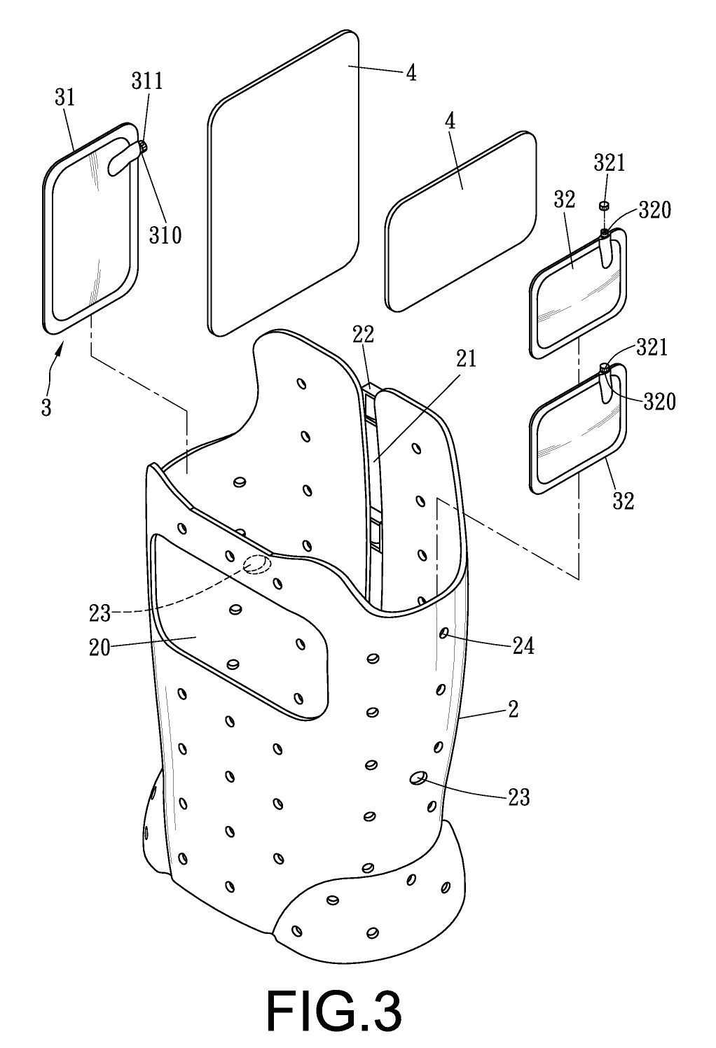

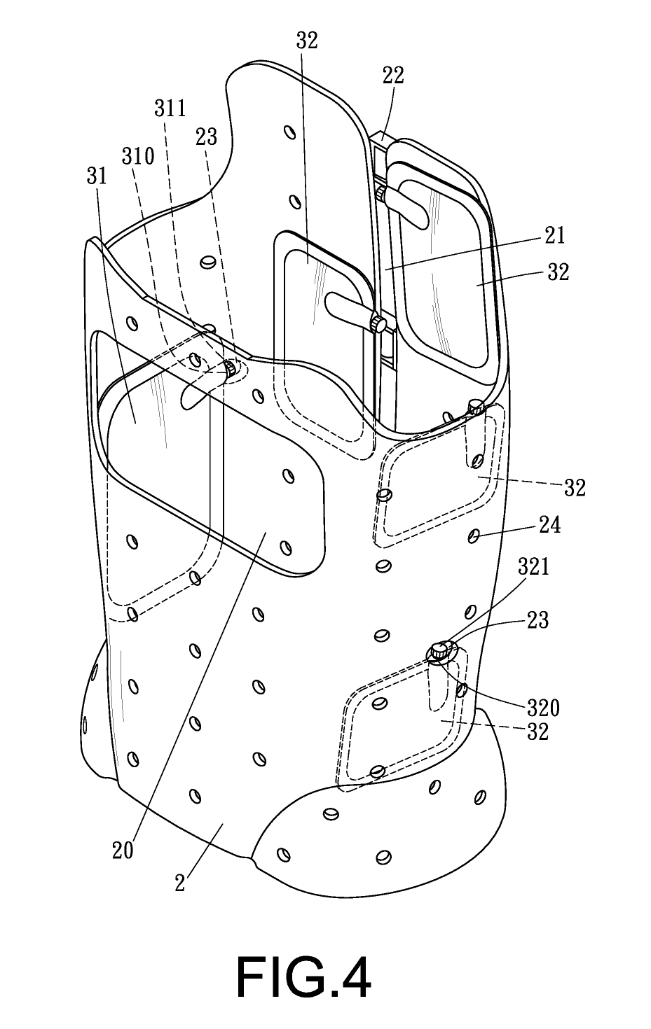

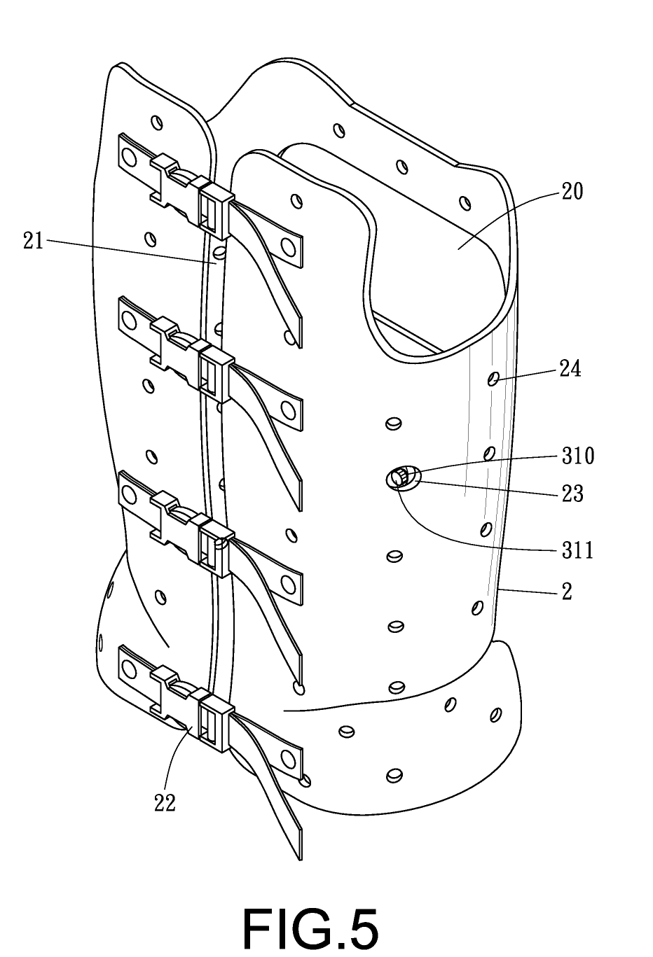

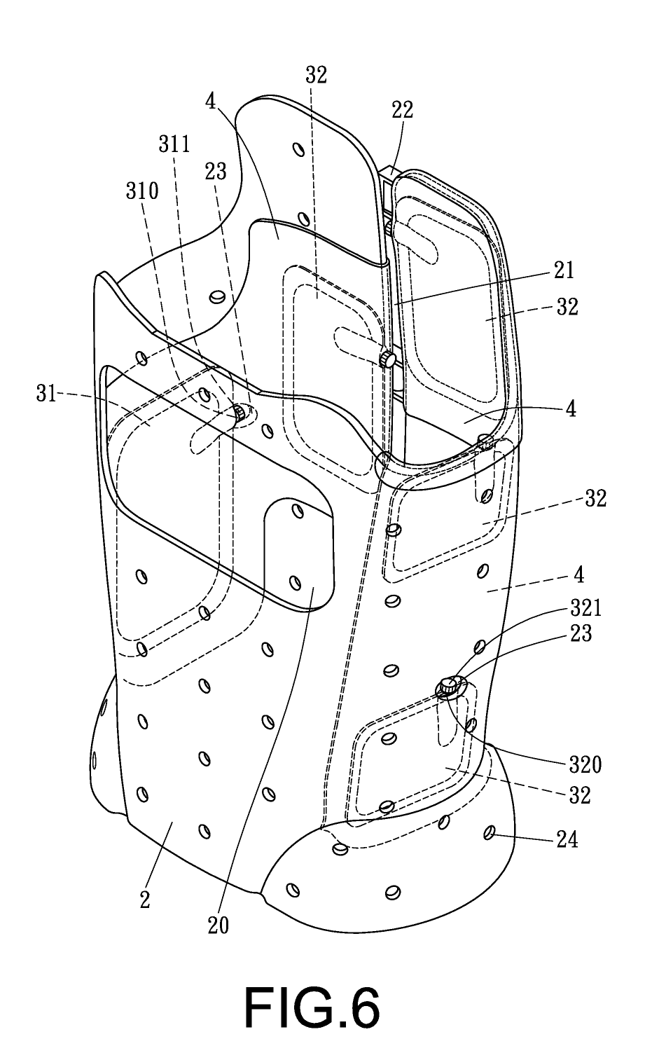

FIGS. 2˜6 show a preferred embodiment of a back brace and its manufacturing method for spinal correction in the present invention. The back brace is composed of a correction brace 2, at least an air bag 3 and at least a protection cushion 4. Before making the correction brace 2, a patient's spine is previously photoed by X-ray to identify its deformity. Then, as shown in FIG. 2A, an original gypseous model (a) is made according to a patient's shape with a curvature. Next, as shown in FIG. 2B, the original gypseous model (a) is amended to become a revised model (A) in a normal shape, with a recessed portion (a1) filled with gypsum and with a humped portion (a2) cut off. The revised model (A) is successively wrapped with a heated thermoplastic board (b), as shown in FIG. 2C. The thermoplastic board (b) is the correction brace 2 after cooled down. The correction brace 2 is provided with a big through hole 20 bored at its front side for corresponding to a user's chest, an vertical opening 21 cut in its rear side for being pulled wide open to enable a patient to wear it on, plural locking members 22 correspondingly positioned at two sides of the vertical opening 21 for adjusting the width of the vertical opening 21 and the tightness of the correction brace 2, and plural small through holes 23 and vents 24 bored spaced apart in it. Plural air bags 3 with different sizes are fixed on the inner wall of the correction brace 2. The air bags 3 include at least a primary air bag 31 and plural secondary air bags 32, each of which is provided with an inflation valve 310 and 320 extending out through the small through hole 23, and a cap 311 and 321 sealed on the inflation valve 310 and 320. The protection cushion 4 is flexible, laid on the air bag 3. As described previously, the back brace is thus finished.

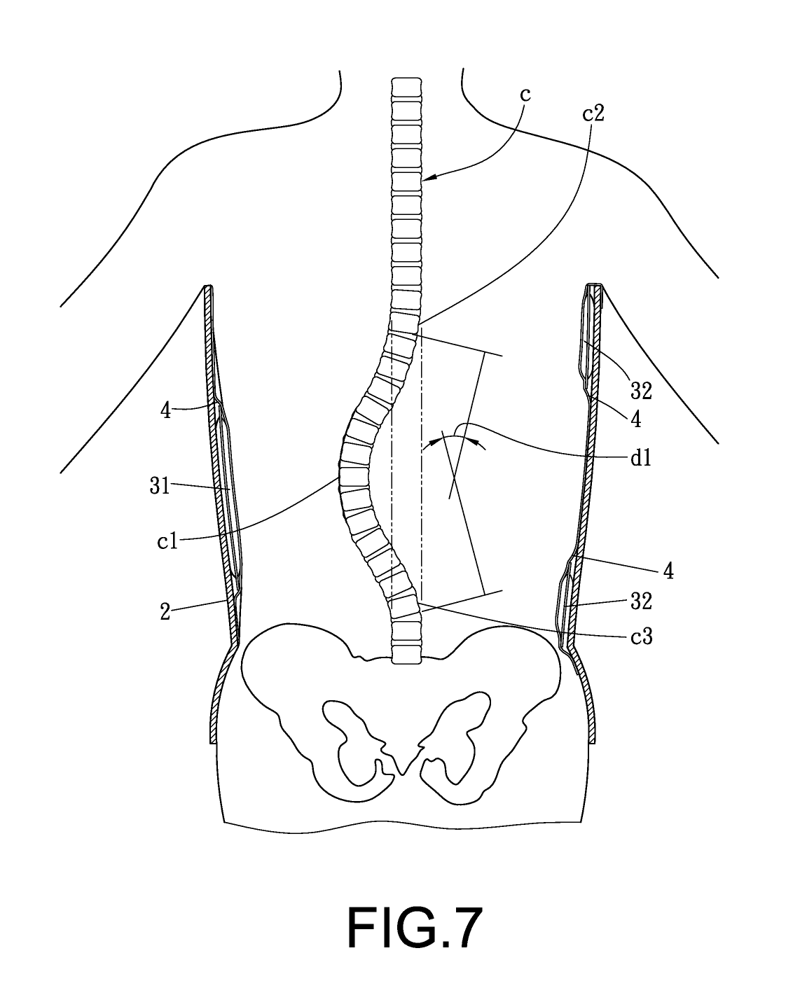

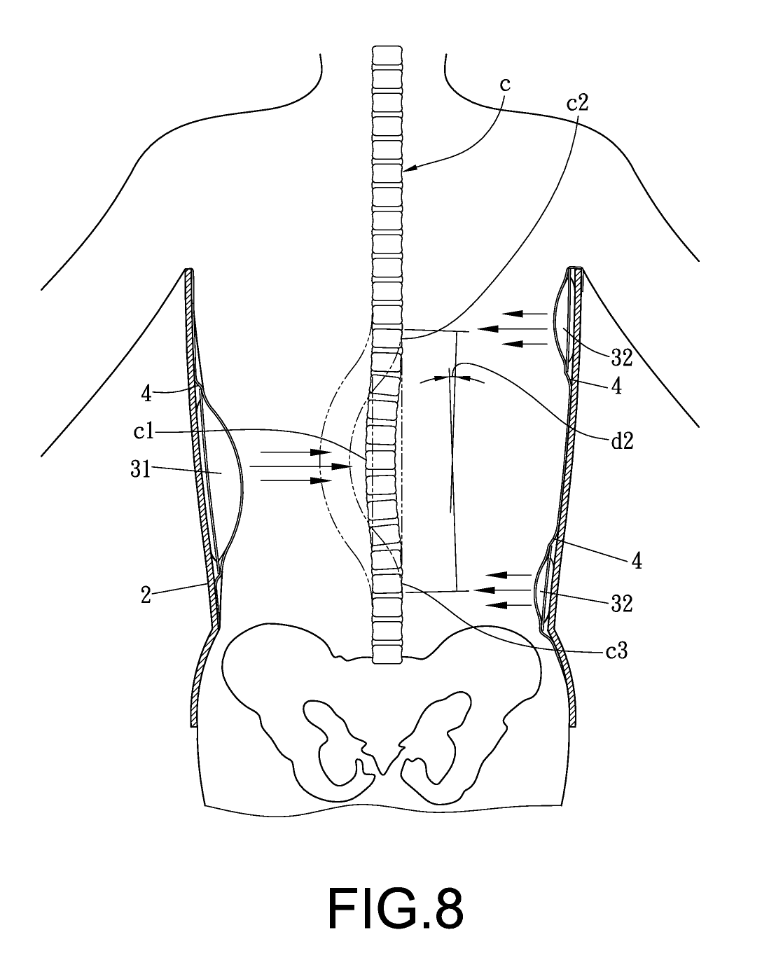

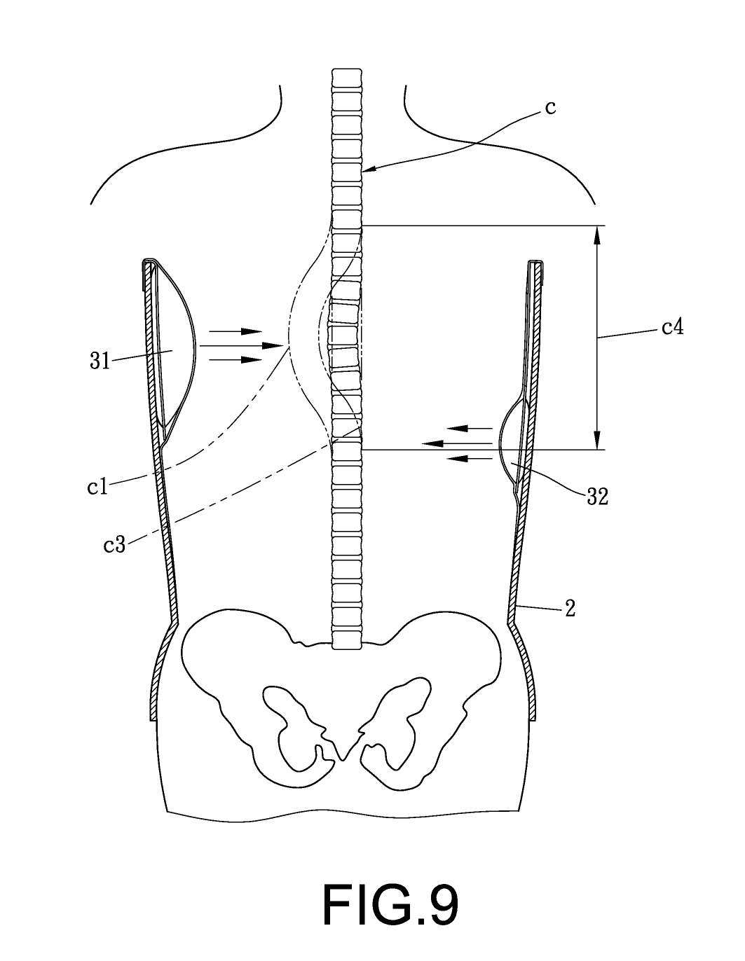

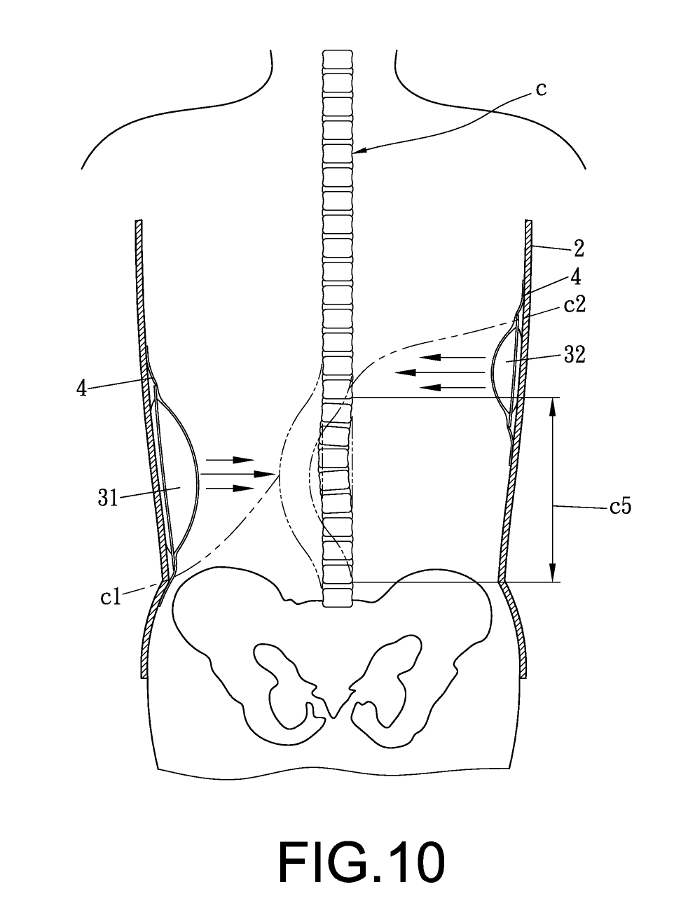

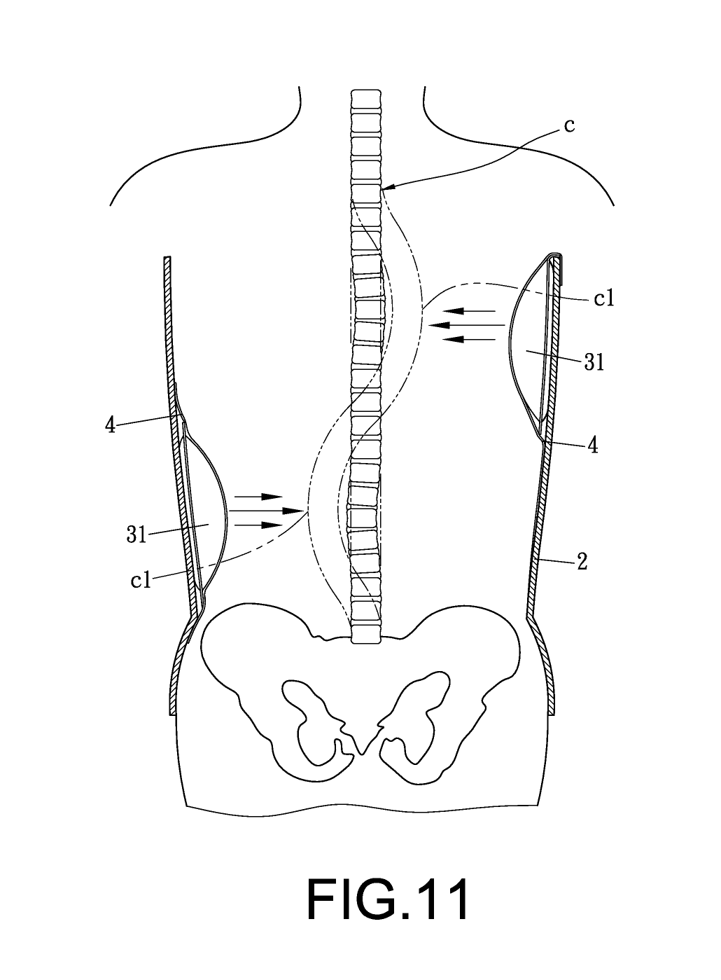

In assembling and using, as shown in FIG. 7, first of all, an original deformation angle (d1) of a patient's spine (c) can be measured according to an X-ray film. Fixed inside the correction brace 2 to correspond to a humping portion (c1) of the spine (c) is the primary air bag 31, and fixed opposite to the primary air bag 31 inside the correction brace 2 to correspond to an upper point (c2) and a lower point (c3)—respectively a starting point and an end point of the original deformation angle (d1)—are the secondary air bags 32. The primary air bag 31 and the secondary air bags 32 are laid with the protection cushions 4 so as to prevent them from contacting with a user's skin, as they may make skin feel uncomfortable. By the time, the correction brace 2 can be worn on a patient, with the primary air bag 31 positioned to corresponding to the humping portion (c1) of the spine (c) of the patient, with the secondary air bags 32 corresponding to the upper point (c2) and the lower point (c3), and with the locking members 22 tightly fastened. Next, through the inflation valves 310 and 320 fitted in the small through holes 23, the primary air bag 31 and the secondary air bags 32 are inflated with a required pressure by a professional. By the time, as shown in FIG. 8, the inflated primary air bag 31 is to powerfully force the humping portion (c1) to move back to or near its original position for greatly shrinking the original deformation angle (d1) to a reduced deformation angle (d2), and the secondary air bags 32 are respectively to force the upper point (c2) and the lower point (c3) with a smaller power so as to prevent the upper point (c2) and the lower point (c3) from being pushed back by the powerful primary air bag 31. If the patient's spine (c) has a humping portion (c1) located around a chest vertebra (c4) as shown in FIG. 9, or a humping portion (c1) located around a waist (c5) as shown in FIG. 10, or has an S-shaped scoliosis (having two humping portions (c1)) as shown in FIG. 11, the primary air bag 31 is employed to correspond to the humping position (c1) and the secondary air bag 32 is used to correspond to the upper point (c2) or the lower point (c3), so as to positively correct the deformities of the spine (c). Of course, the patient must periodically return for checking up the original deformation angle (d1) so as to accordingly adjust the pressure of the primary air bag 31 and the secondary air bags 32. If adjustment is necessary, a professional can directly inflate or deflate the air bags 3 (31 and 32) easily through the inflation valves 310 and 320 fitted in the small through holes 23, without necessity of undressing the correction brace 2. Therefore, according to a diversity of the original deformation angle (d1), the primary air bag 31 and the secondary air bags 32 can be properly pressurized to appropriately force the deformed spine (c), so that the spine (c) can be not only prevented from continuously worsening but practically corrected.

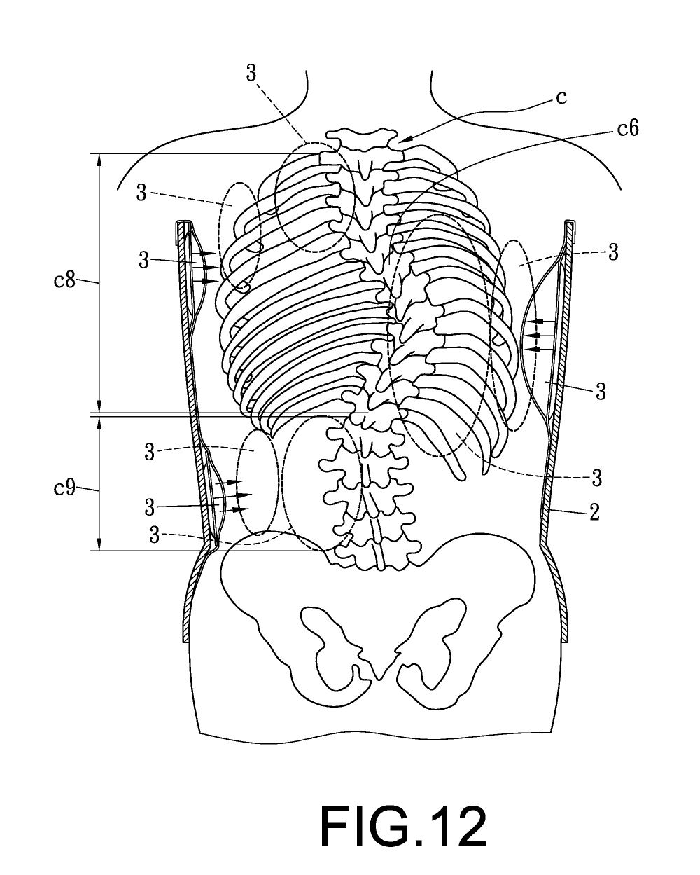

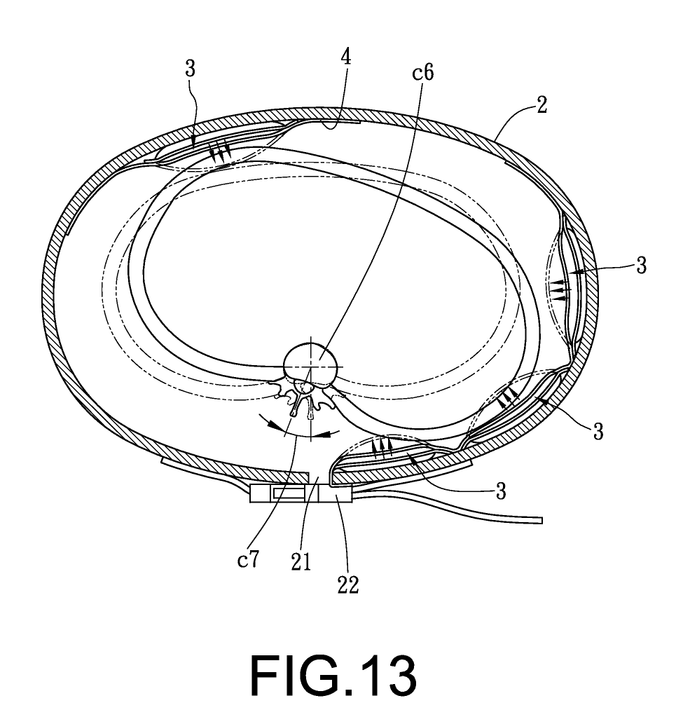

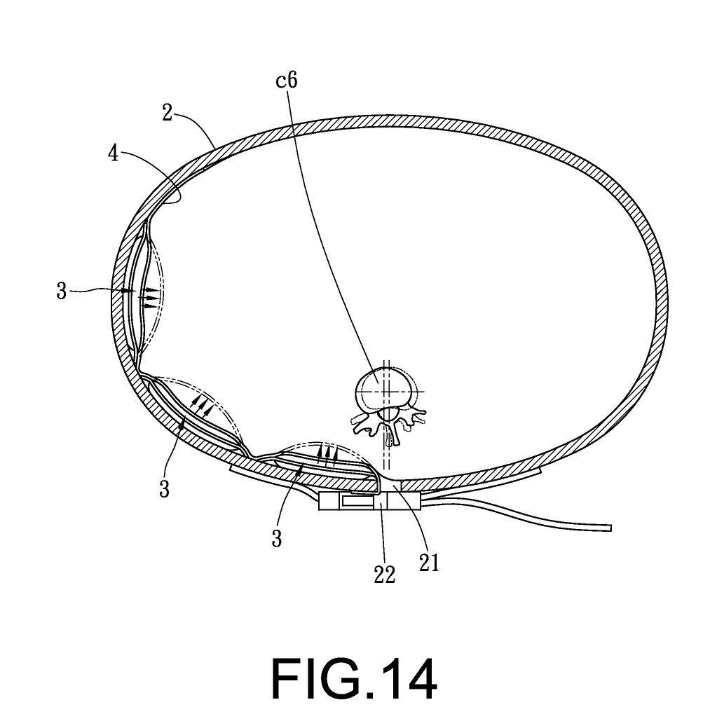

And, FIGS. 12˜14 show a case that the curved spine (c) is coupled with a rotation of its vertebra (c6). As shown in FIG. 12, the air bags 3 can be installed inside the correction brace 2 to correspond to the deformed portion of the spine (c), with the choice of the primary air bag 31 or the secondary air bag 32 depending on the spinal curvature. FIG. 13 and 14 respectively show a top cross-sectional view of a chest vertebra (c8) and a waist vertebra (c9) coupled with the spinal rotation angle (c7). By means of the air bags 3 fixed behind the portion of the spine (c) to be corrected at diverse proper locations, the humping position (c1) of the spine (c) and the rotation angle (c7) of the vertebra (c6) can be forced back to or near their original positions. So, combined with other correction cures and rehabilitation, the scoliosis of the spine (c) coupled with spinal rotation can be effectively corrected by the three-dimensional correction of the back brace.

It is to be noted that when the spine (c) is gradually corrected to move back to its original position, it is to extend upward to enable the height of the patient gradually lengthened. Thus, either the air bags 3 inside the correction brace 2 must have their locations adjusted to adapt the body's change, or the correction brace 2 must be replaced with a new one if the body has changed too much to continuously wear it, so as to achieve a best correction.

The invention has the following advantages as described below.

1. A patient who conventionally needs a surgery to cure a bad scoliosis can wear the back brace to positively have an effective correction for scoliosis and reverse rotation of spine.

2. The back brace can be used by a patient having a scoliosis curve less than 40 degrees.

3. A patient can have his (her) scoliosis corrected by wearing the back brace without necessity of surgery.

4. While adjusting the back brace, a professional can directly inflate or deflate the air bags 3 easily through the inflation valves 310 and 320 fitted in the small through holes 23, without necessity of undressing it.

5. The back brace needs to be only worn 15˜18 hours per day, with 5˜8 hours shorter than 23 hours of the conventional one.

6. Wearing the back brace is not to pose atrophy of the back muscles.

7. As the back brace is worn inside clothes, it is not to make a wearer look clumsy, thus able to attract a scoliosis patient to wear it.

While the preferred embodiment of the invention has been described above, it will be recognized and understood that various modifications may be made therein and the appended claims are intended to cover all such modifications that may fall within the spirit and scope of the invention.

What is claimed is:

1. A back brace for spinal correction and its manufacturing method, a patient's spine previously photoed by X-ray to identify its deformity before making said back brace, an original gypseous model made according to a patient's body shape with a curvature, amending said original gypseous model to become a revised model in a normal shape by filling a recessed portion with gypsum and cutting off a humped portion, said revised model successively wrapped with a heated thermoplastic board that becomes a correction brace after cooled down, said correction brace provided with a vertical opening cut in its rear side for being pulled wide open to enable a patient to wear it on, plural locking member correspondingly positioned at two sides of said vertical opening for adjusting a width of said vertical opening and a tightness of said correction brace, plural air bags having different sizes and fixed on an inner wall of said correction brace to correspond to a humped portion of a scoliosis and an upper point and a lower point of said humped portion and a location of a rotated vertebra, each said air bag provided with an inflation valve fitted in a small through hole bored in said correction brace for a professional to inflate or deflate it and laid with a soft protection cushion, an effective correction of scoliosis and reverse rotation of said vertebra able to be achieved by said air bags correspondingly installed inside said correction brace to force a spinal deformed portion in three dimensions.
